(19) United States
(12) Patent Application Publication
(10) Pub. No.: US 2022/0411525 A1
(43) Pub. Date: Dec. 29, 2022

(54) BCMA AS A TARGET FOR T CELL REDIRECTING ANTIBODIES IN B CELL LYMPHOMAS

(71) Applicants: Janssen Pharmaceutica NV, Beerse (BE); ACADEMISCH MEDISCH CENTRUM, Armsterdam (NL)

(72) Inventors: Homer Adams, III, Elkins Park, PA (US); Raluca Verona, Spring House, PA (US); Arnon P. Kater, Amsterdam (NL); Eric Frederik Eldering, Amsterdam (NL)

(21) Appl. No.: 17/825,123

(22) Filed: May 26, 2022

Related U.S. Application Data
(60) Provisional application No. 63/194,470, filed on May 28, 2021.
(60) Provisional application No. 63/209,694, filed on Jun. 11, 2021.

(51) Int. Cl. C07K 16/28; A61P 35/00; C07K 16/40

(52) U.S. Cl. CPC C07K 16/2878; A61P 35/00; C07K 16/40; A61K 2039/505

(57) ABSTRACT

Provided are methods for treating non-Hodgkin lymphoma (NHL) in a human subject comprising administering to the subject a therapeutically effective amount of a BCMA-specific antibody. Also disclosed are compositions comprising a BCMA-specific antibody in an amount that is therapeutically effective for treating non-Hodgkin lymphoma (NHL) in a human subject, and a γ-secretase inhibitor

### CROSS-REFERENCE TO RELATED APPLICATIONS

The present application claims the benefit of priority to U.S. Provisional Application No. 63/194,470, filed May 28, 2021, and U.S. Provisional Application No. 63/209,694, filed Jun. 11, 2021, the entire contents of both of which are incorporated herein by reference.

### TECHNICAL FIELD

The present disclosure pertains to treatments for B lymphoproliferative disorders.

### BACKGROUND

Treatment of B cell lymphoproliferative disorders such as B non-Hodgkin lymphoma (B-NHL) have improved significantly in recent years. This is mostly due to a rapidly expanding treatment armamentarium that not only contains chemo-immunotherapy but also targeted agents such as inhibitors of pivotal B cell receptor kinases such as BTK and PI3K and inhibitors of key apoptotic regulators such as venetoclax^{1,2}. Nevertheless, for many B-NHL subtypes these treatments are not curative, eventually leading to disease relapse in patients, highlighting the need for new therapies for these malignancies.

From stem cell transplantation (SCT) it has become clear that long-lived T cell mediated anti-cancer responses are feasible³. However, due to severe graft-versus-host disease (GVHD) that can occur, this mostly elderly group of patients are not eligible for SCT. Because application of autologous based T cell therapy arguably will not lead to development of GVHD, it might be a preferable treatment modality. Different autologous based T cell have already been developed, including immune checkpoint blockade (ICB) and chimeric antigen receptor (CAR) T cells. ICB has shown to be effective only in a marginal number of B-NHL patients including follicular lymphoma (FL), diffuse large B cell lymphoma (DLBCL) and mantle cell lymphoma (MCL) patients⁴. In contrast, CAR T cells have proven to be more promising⁴. Currently available CAR T cells are to be produced in a patient-specific manner, making it time-consuming and costly. CAR-T cells are given once and development of T cell exhaustion is a common reason for treatment failure⁵.

In line with the expression of BCMA on PBs and PCs, it is well reported that BCMA is highly expressed on multiple myeloma (MM), and is therefore a suitable target for BCMA targeted therapies¹⁷. Comparable to plasma cells, BCMA signaling also promotes survival of MM cells^{17,18}. However, since expression of BCMA has also been observed on tonsillar memory B cells and germinal center B cells²¹⁻²³, it is unresolved as to whether other mature B cell malignancies also express BCMA and can therefore also be targeted using BCMA-directed therapy.

### SUMMARY

Provided herein are methods for treating non-Hodgkin lymphoma (NHL) in a human subject comprising administering to the subject a therapeutically effective amount of a BCMA-specific antibody.

Also disclosed are compositions comprising a BCMA-specific antibody in an amount that is therapeutically effective for treating non-Hodgkin lymphoma (NHL) in a human subject, and a γ-secretase inhibitor.

### BRIEF DESCRIPTION OF THE DRAWINGS

FIGS. 1A-1E illustrate the results of experiments demonstrating that BCMA is expressed by different B cell malignancies and can be enhanced by γ-secretase inhibition.

FIGS. 2A-2F provide the results of an evaluation showing that BCMA is expressed in low levels on primary CLL cells and can be slightly enhanced by γ-secretase inhibition.

FIGS. 3A-3B illustrate BCMA expression on different B cell malignancies.

FIGS. 4A-4G demonstrate how BCMA antibody induces activation, degranulation, cytokine secretion and cytotoxicity by T cells in the presence of B cell malignancy cell lines.

FIGS. 5A-5C provide the results of an evaluation showing that healthy donor T cells kill primary CLL cells in presence of BCMA-specific antibody, which is largely dependent on CD8+ T cells.

FIGS. 6A-6C illustrate how BCMA-specific antibody induces T cell activation of CLL derived T cells and leads to CLL killing.

FIG. 7 demonstrates that viability of the different cell lines was not affected by γ-secretase inhibition.

FIG. 8 provides data demonstrating that viability CLL cells was not affected by γ-secretase inhibition.

FIGS. 9A-9F provide the results of an assessment regarding whether anti-BCMA antibody treatment, in presence or absence of a γ-secretase inhibitor, would lead to increases in markers of T cell activation (CD25), T cell degranulation (CD107a), cytokine expression (IFN-g, IL-2 and TNF-a) and T cell division.

### DETAILED DESCRIPTION OF ILLUSTRATIVE EMBODIMENTS

The presently disclosed inventive subject matter may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference. In the present disclosure, superscripted numerals refer to the correspondingly numbered publications that appear under the heading “References”, infra.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a treatment” is a reference to one or more of such treatments and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours, etc., up to 5 hours.

B cell maturation antigen (BCMA) is highly expressed on normal and malignant mature B cells, which can be enhanced by inhibition of γ-secretase (y-sec) inhibition, and is a feasible target for multiple myeloma (MM). Data regarding the expression of BCMA on other B cell malignancies is scarce, and for CLL, DLBCL, FL and MCL conflicting data has been published ^{17,24-27}, which has complicated the question whether BCMA is a feasible target in these diseases. Thus, whether BCMA can be targeted by BCMA-specific antibodies such as BCMAxCD3 DuoBody® Teclistamab in other mature B cell malignancies besides MM is currently unknown.

The present inventors evaluated BCMA expression on mature B cell lymphoma cell lines and assessed whether BCMA expression could be enhanced by inhibiting γ-secretase. BCMA expression was also measured and (semi)quantified on primary material from B-NHL (including CLL) patients. To assess whether BCMA can be used as a target in B-NHL (including CLL), it was assessed whether BCMAxCD3 BsAb (Teclistamab, JNJ-7957) could mediate T cell activation as well as tumor cell killing of the lymphoma cell lines. This BsAb has been developed with Genmab DuoBody® technology, resulting in enhanced stability compared to BsAb formats. As a proof-of-concept that BCMA can evoke an autologous T cell response, CLL was used as a target.

The present inventors discovered that BCMA could be variably detected on all tested mature B cell malignancy cell lines, with highest expression in MM, followed by Waldenstrom's macroglobulinemia (WM). In all B cell lines γ-sec inhibition increased BCMA expression, even when basal levels of BCMA were low. These data were corroborated in primary B cell lymphoma, where there were detectable levels of BCMA in samples from WM, CLL, and diffuse large B cell lymphoma patients.

Co-culture of HD T cells with various B cell malignancy cell lines in the presence of Teclistamab resulted in T cell activation, proliferation and cytotoxicity, independent of the level of BCMA expression. Efficacy of teclistamab on primary tumor cells was studied using CLL cells. Despite low BCMA levels, healthy donor T cells lysed CLL cells up to 40% in presence of teclistamab. Furthermore, teclistamab induced activation, degranulation and efficient cytotoxicity by CLL-derived T cells upon coculture with autologous CLL cells.

Accordingly, the present inventors discovered that BCMA expression is not limited to MM, but is also present on other mature B cell malignancies. Targeting BCMA using teclistamab led to cytotoxicity of lymphoma cell lines and primary CLL, even when BCMA levels were low.

In accordance with these discoveries, provided herein are methods for treating non-Hodgkin lymphoma (NHL) in a human subject comprising administering to the subject a therapeutically effective amount of a BCMA-specific antibody.

As used herein, the phrase “therapeutically effective amount” refers to the amount of active compound that elicits the biological or medicinal response that is being sought in a tissue, system, animal, individual or human by a researcher, medical doctor or other clinician, which includes one or more of the following:

(1) at least partially preventing the disease or condition or a symptom thereof; for example, preventing a disease, condition or disorder in an individual who may be predisposed to the disease, condition or disorder but does not yet experience or display the pathology or symptomatology of the disease;

(2) inhibiting the disease or condition; for example, inhibiting a disease, condition or disorder in an individual who is experiencing or displaying the pathology or symptomatology of the disease, condition or disorder (i.e., including arresting further development of the pathology and/or symptomatology); and

(3) at least partially ameliorating the disease or condition; for example, ameliorating a disease, condition or disorder in an individual who is experiencing or displaying the pathology or symptomatology of the disease, condition or disorder (i.e., including reversing the pathology and/or symptomatology).

The BCMA-specific antibody may be monospecific or multispecific (e.g., bispecific), that is, the antibody may be specific to a target other than BCMA, as long as it is also specific to BCMA.

In certain embodiments, the BCMA-specific antibody is a BCMAxCD3 bispecific antibody. Any suitable BCMAxCD3 bispecific antibody known to those skilled in the art in view of the present disclosure can be used in the invention.

Various bispecific antibody formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or bispecific antibodies generated by arm exchange. Exemplary bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), DuoBody (Genmab), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

In some embodiments, the BCMAxCD3 bispecific antibody comprises any one of the BCMA binding domains described in WO2017/031104, the entire content of whch is incorporated herein by reference. In some embodiments, the BCMAxCD3 bispecific antibody comprises any one of the CD3 binding domains described in WO2017/031104. In some embodiments, the BCMAxCD3 bispecific antibody comprises any one of the BCMAxCD3 bispecific antibodies or antigen-binding fragments thereof described in WOb 2017/031104.

In some embodiments, the BCMAxCD3 bispecific antibody comprises a CD3 binding domain comprising a heavy chain complementarity determining region 1 (HCDR1) of SEQ ID NO: 11, a HCDR2 of SEQ ID NO: 12, a HCDR3 of SEQ ID NO: 13, a light chain complementarity determining region 1 (LCDR1) of SEQ ID NO: 14, a LCDR2 of SEQ ID NO: 15 and a LCDR3 of SEQ ID NO: 16; or a heavy chain variable region (VH) of SEQ ID NO: 17 and a light chain variable region (VL) of SEQ ID NO: 18.

In some embodiments, the BCMAxCD3 bispecific antibody comprises a BCMA binding domain comprising a heavy chain complementarity determining region 1 (HCDR1) of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; or a heavy chain variable region (VH) of SEQ ID NO: 7 and a light chain variable region (VL) of SEQ ID NO: 8.

In some embodiments, the BCMAxCD3 bispecific antibody comprises a first heavy chain (HC1) of SEQ ID NO: 9, a first light chain (LC1) of SEQ ID NO: 10, a second heavy chain (HC2) of SEQ ID NO: 19, and a second light chain (LC2) of SEQ ID NO: 20.

In some embodiments, the BCMAxCD3 bispecific antibody is chimeric, humanized or human.

In some embodiments, the BCMAxCD3 bispecific antibody is an antigen binding fragment. Exemplary antigen binding fragments are Fab, F(ab′2, Fd and Fv fragments.

In some embodiments, the bispecific antibody is an IgG1, an IgG2, an IgG3 or an IgG4 isotype. In preferred embodiments, the bispecific antibody is an IgG4 isotype. An exemplary wild-type IgG4 comprises an amino acid sequence of SEQ ID NO: 21.

The bispecific antibody can be of any allotype. It is expected that allotype has no influence on properties of the bispecific antibodies, such as binding or Fc-mediated effector functions. Immunogenicity of therapeutic antibodies is associated with increased risk of infusion reactions and decreased duration of therapeutic response (Baert et al., (2003) Engl J Med 348:602-08). The extent to which therapeutic antibodies induce an immune response in the host can be determined in part by the allotype of the antibody (Stickler et al., (2011) es and Immunity 12:213-21). Antibody allotype is related to amino acid sequence variations at specific locations in the constant region sequences of the antibody. Table 1 shows select IgG1, IgG2 and IgG4 allotypes.

TABLE 1

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)/(n- | T | V | | | | | | |
| nG4m(a) | | | L | R | | | | |
| Glm(17) | | | | | K | E | M | A |
| Glm(17,1) | | | | | K | D | L | A |

In some embodiments, the bispecific antibody comprises one or more Fc substitutions that reduces binding of the bispecific antibody to a Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP). The specific substitutions can be made in comparison to the wild-type IgG4 of SEQ ID NO: 21.

Fc positions that can be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/ L235A on IgG4, N297A on all Ig isotypes, V234A/G237A on IgG2, K214T/E233P/ L234V/L235A/G236-deleted/A327G/P331A/D365E/L358M on IgG1, H268Q/V309L/ A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4, wherein residue numbering is according to the EU index.

Fc substitutions that can be used to reduce CDC are a K322A substitution.

Well-known S228P substitution can further be made in IgG4 antibodies to enhance IgG4 stability.

In some embodiments, the bispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F405L/K409R, wild-type/F405L_R409K, T366Y/F405A, T366W/F405W, F405W/Y407A, T394W/Y407T, T394S/Y407A, T366W/T394S, F405W/T394S and T366W/T366S_L368A Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the BCMAxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the BCMAxCD3 bispecific antibody further comprises proline at position 228, alanine at position 234 and alanine at position 235 in both the HC1 and the HC2.

In some embodiments, the BCMAxCD3 bispecific antibody comprises the HC1 of SEQ ID NO: 9, a first light chain (LC1) of SEQ ID NO: 10, the HC2 of SEQ ID NO: 19 and a second light chain (LC2) of SEQ ID NO: 20.

In some embodiments, the BCMAxCD3 btispecific antibody is CC-93269, BI 836909, JNJ-64007957 (teclistamab), or PF-06863135. In preferred embodiments, the BCMAxCD3 bispecific antibody is teclistamab.

In some embodiments, the amount of teclistamab that is administered to the subject is effective to activate T cells in the subject, induce neutrophil degranulation in the subject, induce cytokine production in the subject, or any combination thereof. In certain embodiments, the amount of teclistamab that is administered to the subject is effective to activate T cells in the subject, induce neutrophil degranulation in the subject, and induce cytokine production in the subject.

The non-Hodgkin lymphoma that is treated pursuant to the present methods may be, for example, any subtype that is characterized by expression of B cell maturation antigen (BCMA). For example, the non-Hodgkin lymphoma may be chronic lymphocytic leukemia (CLL), lymphoblastic lymphoma, mantle cell lymphoma, diffuse large B-cell lymphoma, or Waldenstrom macroglobulinemia. Non-Hodgkin lymphoma is more frequently observed in adults, and so the present methods may include administration of the BCMA-specific antibody to an adult (e.g., an individual over the age of 16). However, non-Hodgkin lymphoma can also occur in children, and the present methods can be used to treat immature humans (individuals that are 16 years of age or younger), as well.

The present methods may further comprise administering to the subject a γ-secretase inhibitor. As described more fully, infra, administration of a γ-secretase inhibitor to the subject can synergize with the BCMA-specific antibody. For example, in order to enhance the efficacy of the BCMA-specific antibody, γ-secretase inhibition could be useful for subjects that express BCMA just below threshold level. As in the case of the administration of the BCMA-specific antibody, the γ-secretase inhibitor is administered in a therapeutically-effective amount, which means that the amount of γ-secretase inhibitor should be administered in an amount that, when the subject is also undergoing treatment with the BCMA-specific antibody, the γ-secretase inhibitor provides a therapeutic effect. The preceding definition of “therapeutically effective amount” applies with respect to the amount of γ-secretase inhibitor when co-administered with the BCMA-specific antibody.

Pursuant to the present methods, treatment with the BCMA-specific antibody may occur at substantially the same time as treatment with a γ-secretase inhibitor. Treatment with BCMA-specific antibody that occurs at substantially the same time as γ-secretase inhibitor refers to situations in which there is temporal overlap between the treatment with BCMA-specific antibody and the γ-secretase inhibitor. Accordingly, treatment with BCMA-specific antibody that occurs during a time period that at least partially overlaps the time period during which administration of the γ-secretase inhibitor occurs can be said to be at substantially the same time. In such instances, the BCMA-specific antibody treatment may commence before or after commencement of the treatment with γ-secretase inhibitor. When there is no overlap between the time period during which treatment with BCMA-specific antibody occurs and the time period during which γ-secretase inhibitor administration occurs, then the treatments may be described as sequential. Thus, in certain embodiments, treatment with BCMA-specific antibody and treatment with γ-secretase inhibitor may occur sequentially. In such instances, the BCMA-specific antibody treatment may commence before or after commencement of the γ-secretase inhibitor therapy.

In some embodiments, the BCMA-specific antibody and the γ-secretase inhibitor are administered to the subject in a single dosage form. Alternatively, the BCMA-specific antibody may be administered in a first dosage form and the γ-secretase inhibitor is administered in a second dosage form.

Also disclosed herein are compositions comprising a BCMA-specific antibody in an amount that is therapeutically effective for treating non-Hodgkin lymphoma (NHL) in a human subject, and a γ-secretase inhibitor. The characteristics and amounts of the BCMA-specific antibody and the γ-secretase inhibitor, respectively (including what constitutes a therapeutically effective amount), may be as described supra in connection with the present methods for treating non-Hodgkin lymphoma.

Pursuant to the presently disclosed methods and compositions, the BCMA-specific antibody, a γ-secretase inhibitor, or both, may be provided in a composition that is formulated for any type of administration. For example, the antibody and/or inhibitor may be provided in a composition (dosage form) that is formulated for administration orally, topically, parenterally, enterally, or by inhalation. In certain embodiments, the composition is formulated for oral administration. The antibody and/or inhibitor may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the antibody and/or inhibitor may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the antibody and/or inhibitor is mixed with a carrier, diluent or excipient having the necessary compression properties in suitable proportions and compacted in the shape and size desired. For oral therapeutic administration, the antibody and/or inhibitor may be incorporated with the carrier, diluent, or excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained.

Liquid carriers, diluents, or excipients may be used in preparing solutions, suspensions, emulsions, syrups, elixirs, and the like. The antibody and/or inhibitor can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy delivery by syringe. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the antibody and/or inhibitor in the pharmaceutically appropriate amounts, in the appropriate solvent, with various of the other ingredients enumerated above, as required, followed by filtered sterilization. Generally, dispersions may be prepared by incorporating the antibody and/or inhibitor into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active agent or ingredients, plus any additional desired ingredient from the previously sterile-filtered solution thereof

The BCMA-specific antibody (and, where applicable, the γ-secretase inhibitor) can be formulated as a pharmaceutical composition comprising about 1 mg/mL to about 200 mg/mL antibody.

In some embodiments, the pharmaceutical composition further comprises one or more excipients. In some embodiments, the one or more excipients include, but are not limited to a buffering agent, a sugar, a surfactant, a chelator, or any combination thereof

In some embodiments, the pharmaceutical composition comprises: about 20 mg/mL to about 120 mg/mL of the BCMA-specific antibody, such as about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, or any value in between, of the BCMA-specific antibody; about 5 mM to about 20 mM buffering agent, such as about 5 mM, about 10 mM, about 15 mM, about 20 mM, or any value in between, sodium phosphate, KH2PO4, sodium acetate or sodium citrate;

about 1% w/v to about 20% w/v sugar, such as about 1% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, or any value in between, glucose, sucrose or cellobiose;

about 0.01% w/v to about 2% w/v surfactant, such as about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.5% w/v, about 1% w/v, about 1.5% w/v, about 2% w/v, or any value in between, polysorbate 80 (PS-80) or PS-20; and

about 5 mM to about 40 mM, such as about 5 mM, about 10 mM, about 15 mM, about 20 mM, about 25 mM, about 30 mM, about 35 mM, about 40 mM, or any value in between, ethylenediaminetetraacetic acid (EDTA) or an edetate salt, at a pH of about 5-6, such as about 5, about 5.1, about 5.2, about 5.3, about 5.4, about 5.5, about 5.6, about 5.7, about 5.8, about 5.9, about 6, or any value in between.

In some embodiments, the pharmaceutical composition further comprises about 0.1 mg/mL to about 5 mg/mL amino acid, such as about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, or any value in between, methionine or arginine.

In one embodiment, a pharmaceutical composition useful for the invention comprises BCMA-specific antibody, such as teclistamab, 20 mM sodium phosphate, 10% weight/volume (w/v) sucrose, 0.06% (w/v) PS80, and 25 μg/mL EDTA at pH 5.4.

In another embodiment, a pharmaceutical composition useful for the invention comprises BCMA-specific antibody, such as teclistamab, 10 to 15 mM sodium acetate, 8% (w/v) sucrose, 0.04% (w/v) PS20, and 20 μg/mL EDTA at pH 5.2.

In another embodiment, a pharmaceutical composition useful for the invention comprises BCMA-specific antibody, such as teclistamab, 15 mM KH2PO4, 10% (w/v) cellobiose, 0.05% (w/v) PS20, and 25 μg/mL EDTA at pH 5.1. Administration

In some embodiments, the BCMA-specific antibody is administered by an intravenous injection.

In some embodiments, the BCMA-specific antibody is administered by a subcutaneous injection.

The dose of the BCMA-specific antibody given to a subject having cancer, such as multiple myeloma, is sufficient to alleviate or at least partially arrest the disease being treated (“therapeutically effective amount”) and includes from about 0.1 μg/kg to about 6000 μg/kg, e.g. about 0.3 μg/kg to about 5000 μg/kg, about 0.1 μg/kg to about 3000 μg/kg, about 0.2 μg/kg to about 3000 μg/kg, about 0.3 μg/kg to about 3000 μg/kg, about 0.6 μg/kg to about 3000 μg/kg, about 1.2 μg/kg to about 3000 μg/kg, about 19.2 μg/kg to about 3000 μg/kg, about 35 μg/kg to about 3000 μg/kg, about 80 μg/kg to about 3000 μg/kg, about 100 μg/kg to about 3000 μg/kg, about 270 μg/kg to about 3000 μg/kg, about 720 μg/kg to about 3000 μg/kg, about 0.1 μg/kg to about 1800 μg/kg, about 0.2 μg/kg to about 1800 μg/kg, about 0.3 μg/kg to about 1800 μg/kg, about 0.6 μg/kg to about 1800 μg/kg, about 1.2 μg/kg to about 1800 μg/kg, about 19.2 μg/kg to about 1800 μg/kg, about 35 μg/kg to about 1800 μg/kg, about 80 μg/kg to about 1800 μg/kg, about 100 μg/kg to about 1800 μg/kg, about 270 μg/kg to about 1800 μg/kg, about 720 μg/kg to about 1800 μg/kg, about 0.1 μg/kg to about 1500 μg/kg, about 0.2 μg/kg to about 1500 μg/kg, about 0.3 μg/kg to about 1500 μg/kg, about 0.6 μg/kg to about 1500 μg/kg, about 1.2 μg/kg to about 1500 μg/kg, about 19.2 μg/kg to about 1500 μg/kg, about 35 μg/kg to about 1500 μg/kg, about 80 μg/kg to about 1500 μg/kg, about 100 μg/kg to about 1500 μg/kg, about 270 μg/kg to about 1500 μg/kg, about 720 μg/kg to about 1500 μg/kg, about 0.1 μg/kg to about 850 μg/kg, about 0.2 μg/kg to about 850 μg/kg, about 0.3 μg/kg to about 850 μg/kg, about 0.6 μg/kg to about 850 μg/kg, about 1.2 μg/kg to about 850 μg/kg, about 19.2 μg/kg to about 850 μg/kg, about 35 μg/kg to about 850 μg/kg, about 80 μg/kg to about 850 μg/kg, about 100 μg/kg to about 850 μg/kg, about 270 μg/kg to about 850 μg/kg, about 720 μg/kg to about 850 μg/kg, about 0.1 μg/kg to about 720 μg/kg, about 0.2 μg/kg to about 720 μg/kg, about 0.3 μg/kg to about 720 μg/kg, about 0.6 μg/kg to about 720 μg/kg, about 1.2 μg/kg to about 720 μg/kg, about 19.2 μg/kg to about 720 μg/kg, about 35 μg/kg to about 720 μg/kg, about 80 μg/kg to about 720 μg/kg, about 100 μg/kg to about 720 μg/kg, about 270 μg/kg to about 720 μg/kg, about 720 μg/kg to about 720 μg/kg, about 0.1 μg/kg to about 270 μg/kg, about 0.2 μg/kg to about 270 μg/kg, about 0.3 μg/kg to about 270 μg/kg, about 0.6 μg/kg to about 270 μg/kg, about 1.2 μg/kg to about 270 μg/kg, about 19.2 μg/kg to about 270 μg/kg, about 35 μg/kg to about 270 μg/kg, about 80 μg/kg to about 270 μg/kg, about 100 μg/kg to about 270 μg/kg, about 270 μg/kg to about 270 μg/kg, about 720 μg/kg to about 270 μg/kg, about 0.1 μg/kg to about 100 μg/kg, about 0.2 μg/kg to about 100 μg/kg, about 0.3 μg/kg to about 100 μg/kg, about 0.6 μg/kg to about 100 μg/kg, about 1.2 μg/kg to about 100 μg/kg, about 19.2 μg/kg to about 100 μg/kg, about 35 μg/kg to about 100 μg/kg, about 80 μg/kg to about 100 μg/kg, about 100 μg/kg to about 100 μg/kg, about 270 μg/kg to about 100 μg/kg, about 720 μg/kg to about 100 μg/kg of the antibody. Suitable doses include, e.g., about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, about 2000 μg/kg, about 2500 μg/kg, about 3000 μg/kg, about 3500 μg/kg, about 4000 μg/kg, about 4500 μg/kg, about 5000 μg/kg, about 5500 μg/kg, about 6000 μg/kg, or any dose in between.

A fixed unit dose of the BCMA-specific antibody can also be given, for example, 50, 100, 200, 500, or 1000 mg, or any value in between, or the dose can be based on the patient's surface area, e.g., 500, 400, 300, 250, 200, or 100 mg/m², or any value in between. Usually 1 to 8 doses, (e.g., 1, 2, 3, 4, 5, 6, 7, or 8) can be administered to treat a cancer, such as a multiple myeloma, but 9, 10, 11, 12, 13, 14, 15, 16, 17, 18, 19, 20, or more doses can be given.

The administration of the BCMA-specific antibody can be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration can be at the same dose or at a different dose. For example, the BCMA-specific antibody can be administered at a first dose at weekly intervals for a certain number of weeks, followed by administration at a second dose every two weeks for an additional certain number of weeks, followed by administration at a third dose every week for an additional certain number of weeks.

The BCMA-specific antibody can be administered by maintenance therapy, such as, e.g., once a week for a period of 6 months or more. For example, the BCMA-specific antibody can be provided as a daily dosage in an amount of about 0.1 μg/kg to about 6000 μg/kg, e.g. about 0.2 μg/kg to about 3000 μg/kg, about 0.2 μg/kg to about 2000 μg/kg, about 0.2 μg/kg to about 1500 μg/kg, about 0.3 μg/kg to about 1500 μg/kg, about 0.6 μg/kg to about 720 μg/kg, about 1.2 μg/kg to about 270 μg/kg, about 19.2 μg/kg to about 720 μg/kg, about 35 μg/kg to about 850 μg/kg, about 270 μg/kg to about 720 μg/kg, of the antibody per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof

In one embodiment, the BCMA-specific antibody is administered intraveneously once a week at a single dose. For example, the BCMA-specific antibody can be administered intravenously once a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, or any dose in between.

In one embodiment, the BCMA-specific antibody is administered intraveneously twice a week at a single dose. For example, the BCMA-specific antibody can be administered intravenously twice a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, or any dose in between.

In one embodiment, the BCMA-specific antibody is administered intraveneously at a step-up (or “priming”) dose, followed by weekly administration at a higher dose. For example, the BCMA-specific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by weekly intravenous administration at a dose of about 35 μg/kg, about 38.4 μg/kg, about 40 μg/kg, about 50 μg/kg, about 57.6 μg/kg, about 60 μg/kg, about 80 μg/kg, or any dose in between.

In one embodiment, the BCMA-specific antibody is administered intraveneously at a step-up dose, followed by administration at a higher step-up dose, followed by weekly administration at a third, higher dose. For example, the BCMA-specific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by intravenous administration at a step-up dose of about 35 μg/kg, about 38.4 μg/kg, about 40 μg/kg, about 50 μg/kg, about 57.6 μg/kg, about 60 μg/kg, about 80 μg/kg, or any dose in between, followed by weekly intravenous administration at a dose of about 80 μg/kg, about 100 μg/kg, about 120 μg/kg, about 180 μg/kg, about 240 μg/kg, about 270 μg/kg, or any dose in between.

In one embodiment, the BCMA-specific antibody is administered intraveneously at a step-up dose, followed by administration at a higher step-up dose, followed by administration at a third, higher step-up dose, followed by weekly administration at a fourth, higher dose. For example, the BCMA-specific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by intravenous administration at a step-up dose of about 35 μg/kg, about 38.4 μg/kg, about 40 μg/kg, about 50 μg/kg, about 57.6 μg/kg, about 60 μg/kg, about 80 μg/kg, or any dose in between, followed by intravenous administration at a step-up dose of about 80 μg/kg, about 100 μg/kg, about 120 μg/kg, about 180 μg/kg, about 240 μg/kg, about 270 μg/kg, or any dose in between, followed by weekly intravenous administration at a dose of about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, or any dose in between.

In one embodiment, the BCMA-specific antibody is administered subcutaneously once a week at a single dose. For example, the BCMA-specific antibody can be administered subcutaneously once a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, about 2000 μg/kg, about 2500 μg/kg, about 3000 μg/kg, about 3500 μg/kg, about 4000 μg/kg, about 4500 μg/kg, about 5000 μg/kg, or any dose in between.

In one embodiment, the BCMA-specific antibody is administered subcutaneously at a step-up dose, followed by weekly administration at a higher dose. For example, the BCMA-specific antibody can be administered subcutaneously at a step-up dose of about 10 μg/kg, about 20 μg/kg, about 35 μg/kg, about 40 μg/kg, about 50 μg/kg, about 60 μg/kg, or any dose in between, followed by weekly subcutaneously administration at a dose of about 80 μg/kg, about 100 μg/kg, about 240 μg/kg, about 300 μg/kg, or any dose in between.

In one embodiment, the BCMA-specific antibody is administered subcutaneously at a step-up dose, followed by administration at a higher step-up dose, followed by weekly administration at a third, higher dose. For example, the BCMA-specific antibody can be administered subcutaneously at a step-up dose of about 10 μg/kg, about 20 μg/kg, about 35 μg/kg, about 40 μg/kg, about 50 μg/kg, about 60 μg/kg, or any dose in between, followed by subcutaneously administration at a step-up dose of about 80 μg/kg, about 100 μg/kg, about 240 μg/kg, about 300 μg/kg, or any dose in between, followed by weekly subcutaneously administration at a dose of about 240 μg/kg, about 720 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, about 2000 μg/kg, about 2500 μg/kg, about 3000 μg/kg, or any dose in between.

In some embodiments, the BCMA-specific antibody is administered for a time sufficient to achieve complete response, stringent complete response, very good partial response, partial response, minimal response or stable disease status, and can be continued until disease progression or lack of patient benefit. The disease status can be determined by any method suitable method known to those skilled in the art in view of the present disclosure, including, e.g., analysis of serum and urine monocolonal protein concentrations, M-protein levels, BCMA levels.

In some embodiments, the BCMA-specific antibody is administered for a time sufficient to achieve complete response that is characterized by negative minimal residual disease (MRD) status. Negative MRD status can be determined by any method suitable method known to those skilled in the art in view of the present disclosure. In some embodiments, negative MRD status is determined using next generation sequencing (NGS). In some embodiments, negative MRD status is determined at 10⁻⁴ cells, 10⁻⁵ cells, or 10⁻⁶ cells.

The BCMA-specific antibody can also be administered prophylactically in order to reduce the risk of developing cancer, delay the onset of the occurrence of an event in cancer progression, and/or reduce the risk of recurrence when the cancer is in remission.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of selinexor, venetoclax, lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, nilotinib, bosutinib, ponatinib, bafetinib, saracatinib, selinexor, venetoclax, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1

### Assessment of BCMA Expression by B Cell Malignancy Cell Lines, and Enhancement by Inhibition of γ-Secretase

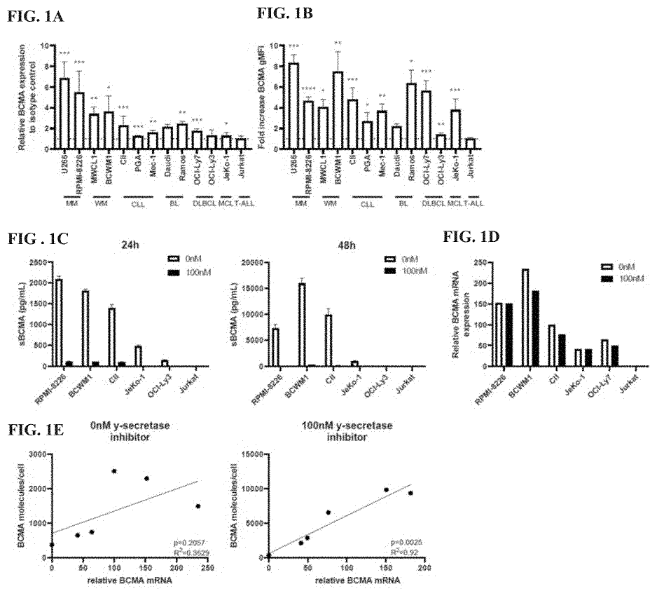

To evaluate whether other malignancies besides multiple myeloma (MM) can potentially be targeted by BCMA directed immunotherapy, B cell malignancy cell lines were assessed for BCMA expression by flow cytometry. FIGS. 1A-1E depict the results of the assessment, by which it was found that BCMA is expressed by different B cell malignancies and can be enhanced by γ-secretase inhibition. Per FIG. 1A, B cell malignancy cell lines were cultured and basal levels of BCMA were assessed by flow cytometry and compared to isotype controls. (n=3-8). Dotted line indicates no increase compared to isotype control. In accordance with FIG. 1B, cell lines were treated for 24-48h with 100nM γ-secretase inhibitor or with medium control and BCMA was assessed by flow cytometry. Values are represented as fold increase compared to 0 nM γ-secretase inhibitor. (n=3-12) Dotted line indicates no increase compared to 0 nM γ-secretase inhibitor. FIG. 1C represents the results of an asssessment of soluble BCMA by ELISA in supernatants of B cell malignancy cell lines after treatment with 0 nM or 100 nM γ-secretase inhibitor for 24-48 h. (n=2). FIG. 1D is an assessment of BCMA mRNA relative to GAPDH control by qPCR after B cell malignancy cell lines were treated without or with 100 nM γ-secretase inhibitor for 24 h. FIG. 1E depicts the correlation between BCMA membrane expression and BCMA mRNA expression in B cell malignancy cell lines without γ-secretase inhibition or with 100 nM γ-secretase inhibition for 24 h. The P value was calculated by paired t test of Wilcoxon test (FIG. 1A-1B) or simple linear regression (FIG. 1E). Data are presented as mean±SD. *P<0.05; **P<0.01; ***P<0.001 ****P<0.0001.

Thus, the MM cell lines U266 (gMFI 5529) and RPMI-8226 (MM, gMFI 4621) expressed high levels of BCMA (FIG. 1A). However, besides MM, high levels of BCMA could be detected on WM cell lines (MWCL1; gMFI 2762 and BCWM.1; gMFI 2069). Lower, but still detectable levels of BCMA were found on cell lines of CLL (CII; gMFI 2059, PGA; gMFI 2097, Mec-1; gMFI 1376), Burkitt lymphoma (Daudi; gMFI 1456 and Ramos; gMFI 1300), DLBCL (OCI-Ly7; gMFI 1086 and OCI-Ly3; gMFI 1177) and MCL (JeKo-1; gMFI 675) (FIG. 1A). As expected, no BCMA was detected on Jurkat cells, which are derived from T cell acute lymphoblastic leukemia (FIG. 1A).

Since BCMA is known to be cleaved off by γ-secretase, it was evaluated whether inhibition of this enzyme would lead to enhanced BCMA levels on these B cell lines. To assess this, the different B cell malignancy lines were incubated for 24-48 h with 100nM γ-secretase inhibitor (Ly411575) and BCMA fold increase was compared to unstimulated cells. Viability of the different cell lines was not affected by γ-secretase inhibition (FIG. 7A). All B cell malignancy cell lines showed an increased BCMA level after γ-secretase inhibition (FIG. 1B). Besides the cell lines which already had high basal levels of BCMA (U266, RPMI-8226, MWCL1 and BCWM.1) also cell lines which had low basal expression of BCMA, such as JeKo-1 and OCI-Ly7 were able to upregulate BCMA after γ-secretase inhibition (FIG. 1B). Again, Jurkat cells showed no upregulation of BCMA, even after γ-secretase inhibition (FIG. 1B). Upregulated levels of BCMA after γ-secretase inhibition suggest active shedding of BCMA by γ-secretase. This was studied by determination of soluble BCMA (sBCMA) levels in supernatants of B cell malignancy cell lines, which were cultured for 24 or 48h in the absence or presence of γ-secretase inhibitor. Indeed, sBCMA levels were detectable upon culture of different selected cell lines (except for Jurkat), and increased upon longer culture times (FIG. 1C). Nevertheless, sBCMA was strongly reduced in addition of the γ-secretase inhibitor at both time points (FIG. 1C), indicating that the observed increase is due to prevention of cleavage. This was further confirmed when assessing mRNA levels, which remained equal prior and after γ-secretase inhibition (FIG. 1D). In line with this, it was observed when quantifying BCMA molecules per cell, that the amount of BCMA per cell showed strong correlation (R²=0.92) with mRNA levels after γ-secretase inhibition (FIG. 1E). This was in contrast without γ-secretase inhibition, which only showed a weak correlation (R²=0.36). Together, these data indicate that besides MM, cell lines derived from different B cell malignancies express BCMA, and such expression can be enhanced by γ-secretase inhibition.

### Example 2

### Assessment of Expression of BCMA by Primary CLL And B Cell Lymphoma Samples

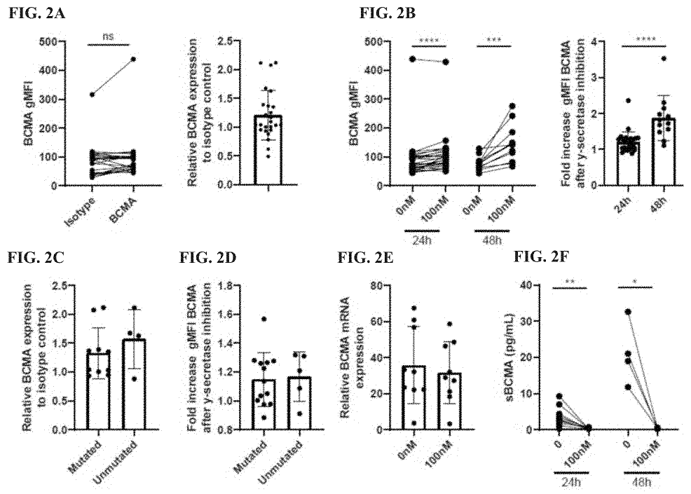

The results regarding BCMA expression on different B cell lymphoma cell lines prompted investigations whether similar results could also be observed on primary material of CLL and B cell lymphoma patients. Primary CLL samples were stained for BCMA expression by flow cytometry and compared to isotype controls. As shown in FIGS. 2A-2F, BCMA is low expressed on primary CLL cells and can be slightly enhanced by γ-secretase inhibition. As provided in FIG. 2A, CLL cells were cultured and basal levels of BCMA were assessed by flow cytometry and compared to isotype controls. (n=25). For FIG. 2B, CLL cells were treated for 24 or 48h with 0nM or 100 nM γ-secretase inhibitor and BCMA was assessed by flow cytometry. Values are represented as fold increase compared to medium control. (n=12-28). FIG. 2C provides basal levels of BCMA compared to isotype control among CLL patients with mutated or unmutated IgVH. (n=4-10). FIG. 2D illustrates the fold increase of BCMA after 24-48 h treatment with 100nM γ-secretase inhibitor compared to medium control among CLL patients with mutated or unmutated IgVH. (n=5-13). FIG. 2E provides the results of an assessment of BCMA mRNA relative to GAPDH control by qPCR after primary CLL samples were treated without or with 100 nM γ-secretase inhibitor for 24 h. (n=9). FIG. 2F provides an assessment of soluble BCMA by ELISA in supernatants of B cell malignancy cell lines after treatment with 100 nM γ-secretase inhibitor or medium control for 24-48 h. (n=4-12) The P value was calculated by Wilcoxon test (FIGS. 2A-2B), Mann Whitney test (FIGS. 2B, 2C, 2D) or paired t test (FIGS. 2E, F). Data are presented as mean±SD. *P<0.05; **P<0.01; ***P<0.001; ****P<0.0001.

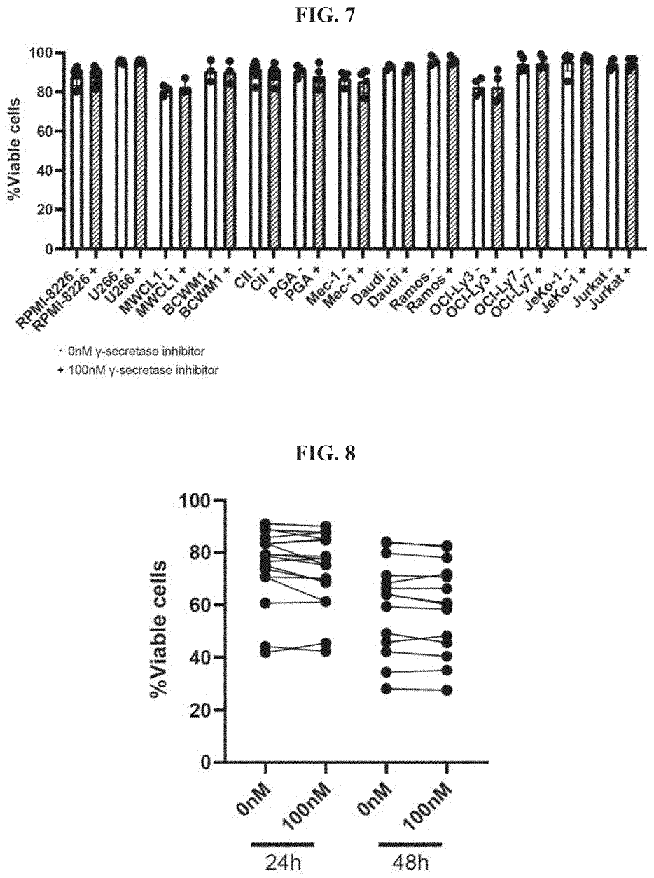

Thus, primary CLL cells showed marginal expression of BCMA (FIG. 2A). Incubation of CLL with a γ-secretase inhibitor for 24 h did lead to a small but significant upregulation of BCMA, which was further enhanced after 48 h (FIG. 2B). Viability of CLL cells was not affected by the inhibitor (FIG. 8A). No difference in BCMA levels, before or after γ-secretase inhibitor treatment, was observed between CLL samples with mutated and unmutated immunoglobulin heavy chain variable region gene (IgHV) status (FIG. 2C-D). Low but measurable mRNA levels of BCMA expression could be detected on CLL cells (FIG. 2E). Despite low BCMA detection by flow cytometry, sBCMA could be detected in supernatants of CLL cells, already after 24 h of culturing, which increased after 48 h (FIG. 2F). Treatment with γ-secretase inhibitor led to marked decreases in sBCMA levels, indicating active shedding of BCMA off these CLL cells (FIG. 2F).

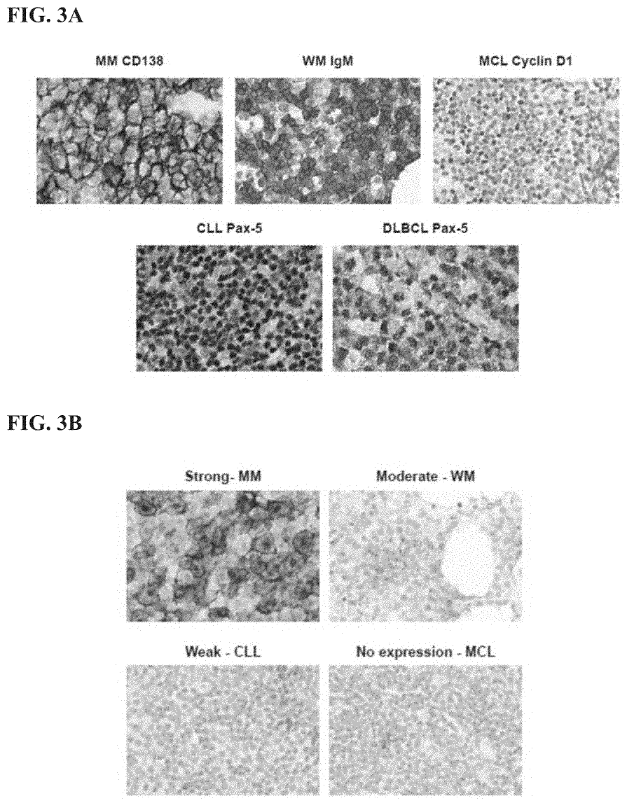

To assess whether BCMA could be detected in LN of CLL patients, BCMA IHC was performed. BCMA expression by IHC was also assessed on primary material on bone marrow or lymph nodes of MM, WM, DLBCL, and MCL patients. FIGS. 3A-3B depict BCMA expression on different B cell malignancies, and show the immunohistochemistry of paraffin embedded slides of different B cell malignancies at 400× magnification. In FIG. 3A, tumor cells were identified per disease type based on staining of Pax-5 for CLL (n=4) and DLBCL (n=3), IgM for WM (n=3), Cyclin D1 for MCL (n=3) and CD138 for MM (n=4). FIG. 3B represents examples of strong, moderate, weak and no expression of BCMA by IHC both on membrane and golgi.

Thus, to determine the amount of tumor cells expressing BCMA, tissues were also stained for CD138 (MM), IgM (WM), Cyclin D1 (MCL) and Pax-5 (CLL and DLBCL) (FIG. 3A). BCMA expression was categorized based on intensity of the staining of both the membrane and Golgi complex (FIG. 3B). The results of the different B cell lymphomas and CLL are summarized in Table 2, below.

TABLE 2
BCMA expression by IHC on different B cell malignancies. Per tumor
type, the amount of tumor cells was determined. BCMA positivity either on membrane or in
golgi was determined as percentage of total tumor cells. Percentages were assessed
independently by two pathologists.

| | | | % BCMA + | BCMA | |
|---|---|---|---|---|---|
| | | % TUMOR | OF TUMOR | MEMBRANE | BCMA GOLGI |
| DISEASE | N | CELLS | CELLS | INTENSITY | INTENSITY |
| MM | 4 | 70-98 | 60-90 | Absent to strong | Weak to strong |
| WM | 3 | 20-65 | 10-90 | Weak to moderate | Weak to moderate |
| DLBCL | 3 | 7-90 | 0-8 | Absent to moderate | Absent to moderate |
| CLL | 4 | 90-95 | 0-0.5 | Absent to weak | Absent to weak |
| MCL | 3 | 80-90 | 0 | Absent | Absent |

Bone marrow biopsy samples of MM patients showed strongest BCMA expression on the tumor cells, either as golgi staining or membrane expression. Also, in bone marrow samples of patients with WM, BCMA could readily be detected. In LN biopsy specimens of CLL and DLBCL patients, BCMA expression was weaker and in LN samples obtained from MCL patients, no BCMA could be detected. These results indicate that BCMA can be expressed on other B cell malignancies besides MM. However, expression was lower, and in some cases, only confined to a small proportion of tumor cells.

### Example 3

### Co-Culture of Healthy Donor PBMCs with B Cell Malignancy Cell Lines in the Presence of BCMA-Specific Antibody

Since the different B cell malignancy cell lines express BCMA to different extents, it was explored whether co-culture of these cell lines with the BCMAxCD3 BsAb Teclistamab in presence of HD PBMCs would lead to activation of T cells. To assess this, 4 cell lines were selected based on previously determined BCMA levels: RPMI-8226 (MM, positive control; high BCMA), BCWM.1 (WM, high BCMA), CII (CLL, intermediate BCMA) and JeKo-1 (MCL, low BCMA). Cell lines and age-matched HD PBMCs were cultured in presence of 100 ng/mL Teclistamab or the control BsAbs (BCMAxnull or nullxCD3) with or without 100 nM γ-secretase inhibitor. As a positive control, anti-CD3/CD28 antibodies were added to the co-cultures to induce TCR stimulation.

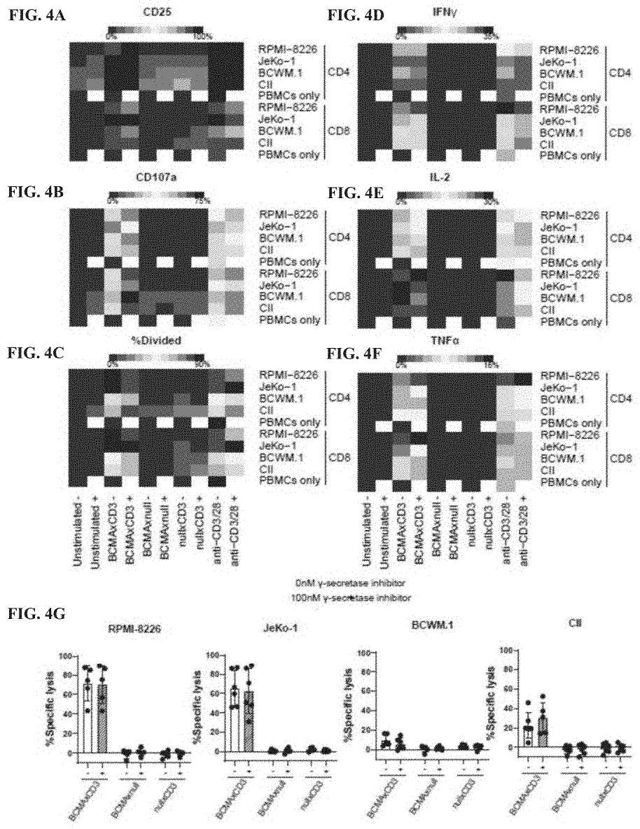

FIGS. 4A-4G provide the results of the assessment, which are that BCMAxCD3 DuoBody® induces activation, degranulation, cytokine secretion and cytotoxicity by T cells in the presence of B cell malignancy cell lines. PBMCs of healthy donors were left unstimulated or stimulated with 100 ng/mL BCMAxCD3 DuoBody®, BCMAxnull, nullxCD3 or anti-CD3/CD28 antibodies. Cells were left untreated (−) or treated with 100nM γ-secretase inhibitor (+). T cells were co-cultured with cell lines RPMI-8226 (multiple myeloma), JeKo-1 (mantle cell lymphoma), BCWM.1 (Waldenstrom's macroglobulinemia) or CII (chronic lymphocytic leukemia) in a 1:1 E:T ratio. After 48 hours activation by CD25 (FIG. 4A), degranulation (FIG. 4B), secretion of IFNy (FIG. 4D), IL-2 (FIG. 4E), TNFα (FIG. 4F), and cytotoxicity (FIG. 4G) were measured by flow cytometry (n=3-14). 4 days after incubation T cell proliferation was assessed by FACS (FIG. 4C) (n=3-9).

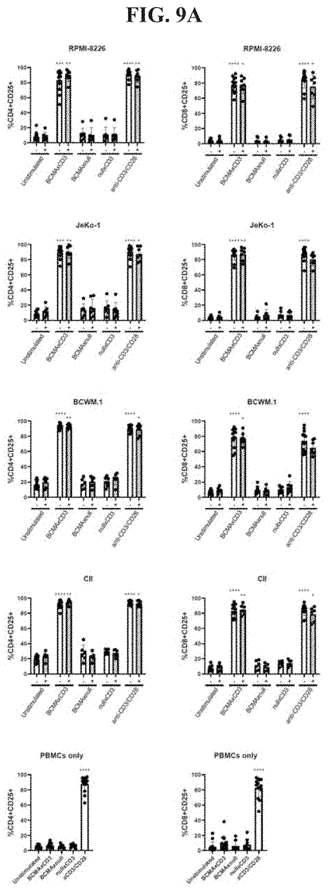

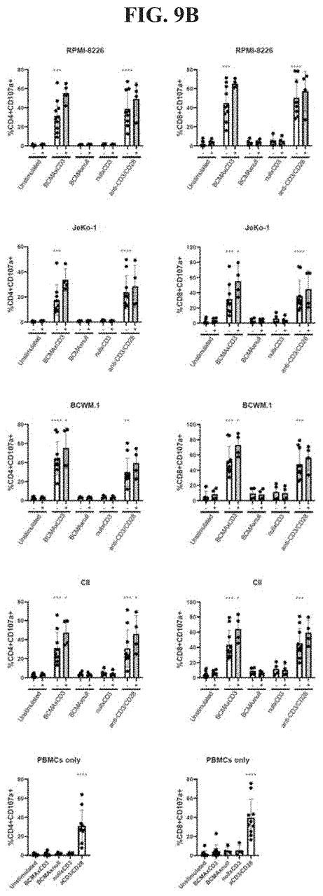

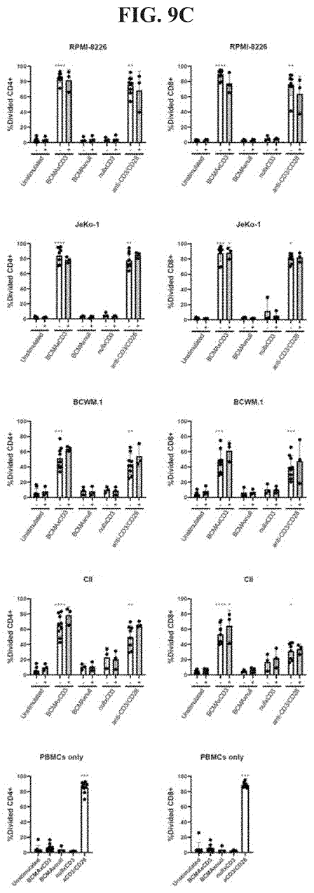

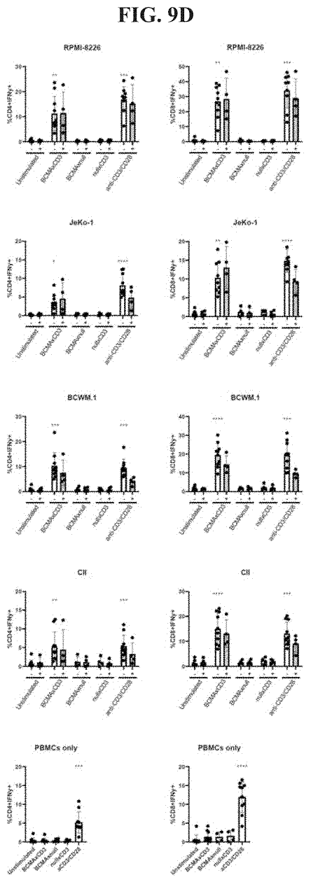

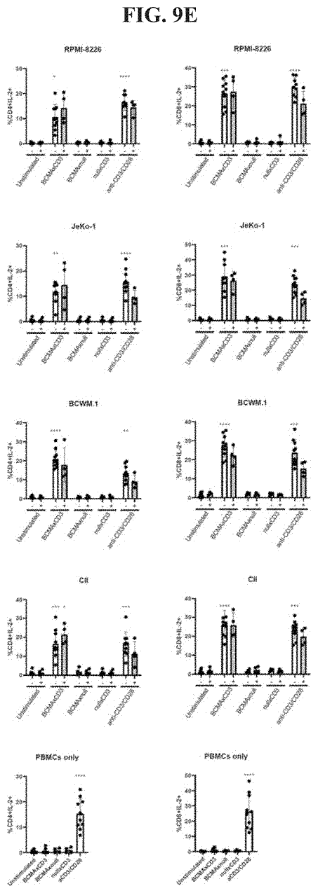

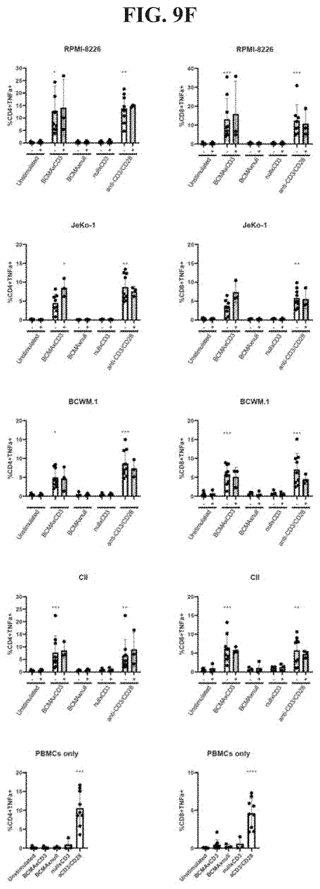

Thus, both CD4⁺ and CD8⁺ T cells showed upregulation of the activation marker CD25 (IL-2 receptor) after 2 days of co-culture with the different cell lines in presence of either Teclistamab or anti-CD3/CD28 stimulation (FIG. 4A and FIG. 9A). No activation was observed using the control BsAbs and when PBMCs were cultured without a target cell, addition of Teclistamab did not lead to upregulation of CD25 (FIGS. 4A and 9A). Similar upregulation was observed after 24 h for CD107a, IFNγ, IL-2 and TNFα (FIG. 4B, 4D-F, 9B and 9D-F). Activation and proliferation did not depend on BCMA expression density, since low BCMA-expressing cells like JeKo-1 induced activation to similar levels as high BCMA-expressing cell line RPMI-8226, and was not further enhanced by increasing BCMA levels by γ-secretase inhibition (FIG. 4A, 4C and FIG. 9A, 9C). Besides T cell activation, degranulation and cytokine production, Teclistamab also induced cell death of target cells upon co-culture with HD T cells (FIG. 4G). Again, cytotoxic potential did not seem to be dependent on BCMA levels, since JeKo-1 was more efficiently lysed than higher BCMA-expressing BCWM.1 or CII cell lines and since it did not improve upon addition of an γ-secretase inhibitor (FIG. 4G). It therefore seems that for Teclistamab activity a certain (low) threshold level of BCMA is necessary to induce proper T cell activation and cytotoxicity. However, these results also show that tumor intrinsic factor may also negatively impact response to teclistamab.

### Example 4

### Despite Low Expression of BCMA by CLL Cells, BCMA-Specific Antibodies Induce Potent Lysis of CLL Cells

The present show that a low amount of BCMA expression can be sufficient to confer for sensitivity of cell lines to Teclistamab. Since primary CLL samples express BCMA at even lower levels compared to JeKo-1 cell lines, the present inventors explored whether this expression level was still high enough to induce effective lysis of CLL cells. To assess this HD T cells were co-cultured for 48-96 h with primary CLL cells in the presence of 100 ng/mL of Teclistamab with or without 100 nM γ-secretase inhibitor.

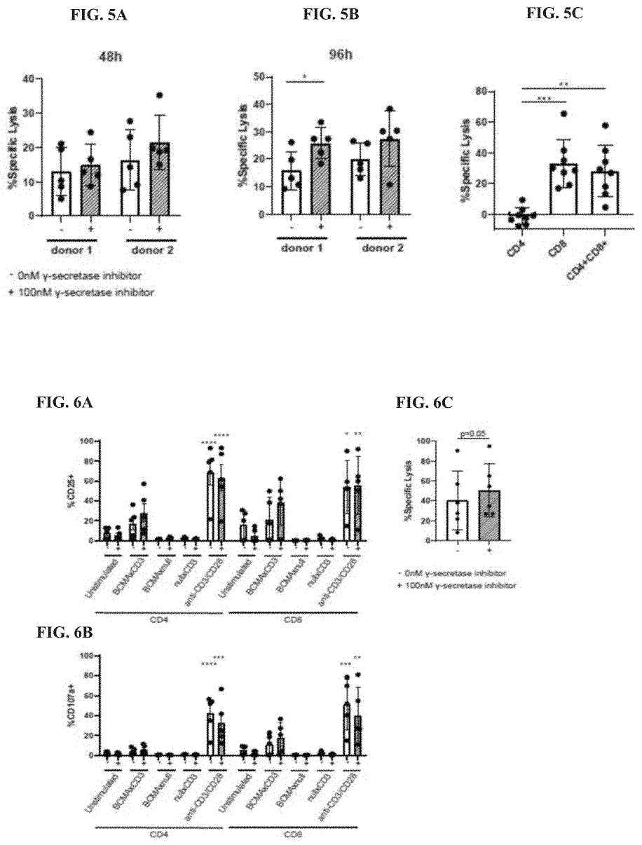

FIGS. 5A-5C illustrate how healthy donor T cells kill primary CLL cells in presence of BCMAxCD3 DuoBody, which is largely dependent on CD8⁺ T cells. Measurement of cytotoxicity after PBMCs of healthy donors were left unstimulated or stimulated with 100 ng/mL BCMAxCD3 DuoBody®, in the absence (−) or presence (+) of 100 nM γ-secretase inhibitor. PBMCs were T cells were co-cultured with primary CLL in a 10:1 E:T ratio for (FIG. 5A) 48 h or (FIG. 5B) 96 h. (n=5) FIG. 5C shows the results of a measurement of cytotoxicity of primary CLL cells that were co-cultured with CD4⁺ or CD8⁺ or CD4⁺ and CD8⁺ (1:1 ratio) in a 5:1 E:T ratio for 96h in the presence or absence of 100 ng/mL BCMAxCD3 DuoBody, and were left untreated (−) or treated with 100nM γ-secretase inhibitor (+). (n=8) The P value was calculated by Wilcoxon test (FIG. 5A), paired t test (FIG. 5B) or repeated measures one-way ANOVA (FIG. 5C). Data are presented as mean±SD. *P<0.05; **P<0.01; ***P<0.001.

Accordingly, after 48 h, induction of cell death could be observed in the CLL cells with average lysis of 12.9% and 16.4% in both T cell donors. This level increased to 14.9% and 21.6% on average upon treatment with γ-secretase inhibitor (FIG. 5A). After 96h the amount of cell death slightly increased to 15.8% and 20% on average for both T cell donors and was 25,8% and 27.4% upon γ-secretase inhibitor treatment (FIG. 5B). Whereas in the cell line data, inhibition of γ-secretase did not lead to enhanced killing, this trend could be observed in CLL, although this did not reach significance in all T cell donors or on all time points (FIG. 5A-B). Contribution of CD4 and/or CD8 was examined by co-cultured of CLL cells with either HD CD4⁺ or CD8⁺ or CD4⁺ and CD8⁺ together (1:1 ratio) in the presence of Teclistamab for 96 h. Surprisingly, CD4⁺ T cells were not able to induce killing of the CD8⁺ T cells, which was in sharp contrast to CD8⁺ T cells, which could induce lysis to up to 60% (FIG. 5C). In contrast to HD T cells, T cells derived from CLL patients are known to be dysfunctional regarding activation, degranulation, synapse formation and cytotoxicity among others²⁸⁻³⁰.

It was also assessed whether Teclistamab would induce activation and cytotoxicity of CLL derived T cells. To assess activation and degranulation, full PBMCs of CLL patients were treated with 100 ng/mL Teclistamab or control BsAbs for 96 h with or without presence of γ-secretase inhibition. FIG. 6A-6C demonstrate how BCMAxCD3 DuoBody induces T cell activation of CLL derived T cells and leads to CLL killing. For FIGS. 6A-6, CLL PBMCs were stimulated with 100 ng/mL BCMAxCD3, BCMAxnull, nullxCD3 or anti-CD3/CD28 antibodies. Flow cytometry analysis of (FIG. 6A) CD25 and (FIG. 6B) CD107a were performed after 4 days (n=3-5). For FIG. 6C, T cells from CLL patients were isolated and co-cultured in a 5:1 E:T ratio with their autologous CLL for 96 h in the presence or absence of 100 ng/mL BCMAxCD3 DuoBody and were left untreated (−) or treated with 100 nM γ-secretase inhibitor (+). (n=6) The P value was calculated ordinary one-way ANOVA (FIGS. 6A-6B) or paired t test (FIG. 6C). Data are presented as mean±SD. *P<0.05; **P<0.01; ***P<0.001; ****P<0.0001.

Accordingly, a trend towards increased CD25 activation could be observed in both CD4⁺ and CD8⁺ T cells of CLL patients in presence of Teclistamab, which is enhanced by addition of γ-secretase inhibitor (FIG. 6A) whereas no upregulation could be detected upon addition of the control BsAbs. Similar results were obtained when assessing degranulation (measured by CD107a), although this was more pronounced in CD8⁺ T cells. Finally, when co-culturing CLL derived T cells with their autologous CLL cells for 96 h in the presence of Teclistamab resulted in mean lysis of 40%, which was slightly increased upon addition of γ-secretase inhibition (FIG. 6C). These results imply that despite low BCMA expression on primary CLL cells, these cells can be efficiently lysed by Teclistamab upon co-culture with CLL derived T cells.

### Materials and Methods

The following materials, conditions, and methods were used pursuant to the experimental work described in Examples 1-3, above.

Patients and controls. Peripheral blood mononuclear cells (PBMCs) were isolated from peripheral blood of CLL patients or buffy coats of (age-matched) healthy donors (HD) from Sanquin Blood Supply (Amsterdam, The Netherlands) using Ficoll-Plaque (VWR). All samples were cryopreserved in liquid nitrogen and CLL samples used had a purity of CD5⁺ CD19⁺ of at least 85%. Paraffin-embedded bone marrow and lymph node tissue (bone marrow from MM and Waldenstrom macroglobulinemia (WM) and lymph node (LN) from DLBCL, MCL and CLL) was obtained from the pathology department of the Amsterdam University Medical Centers, location AMC. Written informed consent was obtained from all subjects in accordance with the Declaration of Helsinki and the study was approved by the medical ethics committee at Amsterdam UMC (ethics approval number 2013/159).

Bispecific antibodies. Full BCMAxCD3 DuoBody (JNJ-7957, JNJ-64007957) and controls BCMAxnull (BC3B4) and nullxCD3 (CNTO7008) were provided by Janssen Pharmaceuticals.

Culture conditions. CLL cells, RPMI-8226, MWCL1, BCWM1, Mec-1, Ramos, OCI-Ly7 and Jurkat cells were cultured in Iscove's Modified Dulbecco's Medium (IMDM, Thermo Fisher Scientific). HD or tonsil derived PBMCs, U266, CII, PGA-1, Daudi, OCI-Ly3 and JeKo-1 cells were cultured in RPMI 1640 medium (Thermo Fisher Scientific). Medium was supplemented with 10% fetal calf serum and 1% penicillin/streptomycin.

Flow cytometry. PBMCs were washed with PBA (PBS, 0.5% BSA and 0.02% Sodium Azide) and stained using fluorescently labelled antibodies for 20 minutes on ice. The following antibodies were used: BCMA APC, BCMA PE (Biolegend), IgG2a kappa isotype PE (BD Biosciences), CD3 V500 (BD Biosciences), CD4 BV605 (BD Biosciences), CD4 PerCPefl710 (eBioscience), CD5 PE (eBioscience), CD5 PerCPCy5.5 (Biolegend), CD8 BV510 (Biolegend), CD8 PECy7 (eBioscience), CD19 APC (BD Biosciences), CD19 FITC (BD Biosciences), CD20 FITC (BD Biosciences), CD25 APC (BD Biosciences), CD25 BV786 (BD Biosciences), CD27 PerCPefl710 (eBioscience), CD38 PE (BD Biosciences), CD38 BV421 (Sony), CD45RA BV650 (Biolegend), CD107a PECy7 (BD Biosciences), CD138 FITC (Molecular Probes), CCR7 BUV395 (BD Biosciences), IgD PE-CF594 (BD Biosciences), IFNy BV421 (BD Biosciences), IL-2 PE-Dazzle594 (Biolegend), TNFα AF700 (BD Biosciences). To exclude dead cells, Fixable Viability Dye eFluor 780 was used according to manufacturer's instructions. For staining of intracellular cytokines, cells were fixed and permeabilized using the Fixation/Permeabilization Solution Kit (BD Biosciences). After antibody staining, samples were washed using PBA and acquired on BD FACS Canto or LSR Fortessa flow cytometer and analysed with FlowJo v10. To quantify number of cells using flow cytometry, 123count eBeads™ Counting Beads (Thermo Fisher Scientific) according to manufacturer's instructions. BCMA molecules per cell were determined by usage of PE Phycoerythrin Fluorescence Quantitation Kit (BD Biosciences).

BCMA characterization by flow cytometry and quantitative polymerase chain reaction. Cell lines or CLL cells were cultured either in medium or in presence of 100 nM γ-secretase inhibitor (Ly411575, Sigma). After 24 or 48 h BCMA was detected by flow cytometry as described above. Relative expression was calculated compared to isotype controls. For qPCR total RNA was isolated using the RNeasy mini kit (Qiagen) and cDNA was transcribed by RevertAid (Fermentas), using random hexamer primers (Promega). qPCRs were performed using SYBR Green master mix (Applied Biosystems) and measured on a Quantstudio 3 (Applied Biosystems). Expression of BCMA was normalized to GAPDH. Linear regression software was used for analysis.

Cytotoxicity assay. Cell lines or primary CLL samples were labelled with Cell Trace Violet (CTV, Thermo Fisher Scientific) or carboxyfluorescein diacetate succinimidyl ester (CFSE, ThermoFisher Scientific) according to manufacturer's instructions and co-cultured with healthy donor PBMCs or CLL derived (autologous) T cells in different effector-target (E:T) ratios. Where indicated prior to co-culture CD4 and CD8 T cells were isolated using MACS beads (Miltenyi), according to manufacturer's instructions. Co-cultures were in the presence of 100 ng/mL BCMAxCD3, BCMAxnull or nullxCD3. 100nM γ-secretase inhibitor (Ly411575) was added where indicated. Viability of the target cells was assessed using TO-PRO-3 (Invitrogen) and MitoTracker Orange (Invitrogen) using Flow Cytometry. Specific lysis of target cells was calculated as (% target cell death in treated sample—% cell death target cells in medium control)/(100—% cell death target cells in medium control)*100%. Samples were excluded when cell death in medium controls exceeded 50%.

T cell proliferation. PBMCs from HD patients were labelled with CTV and cultured alone or in 1:1 E:T ratio with RPMI-8226, JeKo-1, CII or BCWM1. PBMCs were incubated in the presence of 100 ng/mL BCMAxCD3, BCMAxnull or nullxCD3 or stimulated with CD3 (clone 1XE) and CD28 (clone 15E8) antibodies. 100 nM γ-secretase inhibitor (Ly411575) was added where indicated. After 4 days proliferation was measured by flow cytometry as described above.

Activation, cytokine production and degranulation. PBMCs from HD or CLL patients were incubated in the presence of 100 ng/mL BCMAxCD3, BCMAxnull or nullxCD3 or stimulated with CD3 (clone 1XE) and CD28 (clone 15E8) antibodies for 2 days. Where indicated HD PBMCs were co-cultured in a 1:1 E:T ratio with RPMI-8226, JeKo-1, CII or BCWM1. 100 nM γ-secretase inhibitor (Ly411575) was added where indicated. Brefeldin A (10 ug/mL, Invitrogen), GolgiStop (BD Biosciences) and anti-CD107a PE-Cy7 were added 4-6 hours before assessment of activation, degranulation, and cytokine production by flow cytometry as described above.

sBCMA ELISA. Cell lines or CLL cells were cultured either in medium or in presence of 100 nM γ-secretase inhibitor (Ly411575, Sigma). After 24 or 48 h supernatants were harvested and stored at −20° C. soluble BCMA (sBCMA) in supernatants were measured by ELISA using antibody pairs for BCMA.

BCMA immunohistochemistry. IHC stainings for BCMA (clone E6D7B, cell signalling), CD138, Pax-5, Cyclin D1 and IgM were performed on paraffin-embedded bone marrow and LN tissue. Staining were performed by PhenoPath Laboratories (Seattle, Wash.) on a Dako Autostainer EQ240 system. Results were assessed by two independent pathologists.

Statistical analysis. Data was checked for normality by a D'Agostino-Pearson test or if n<5 a Shapiro-Wilk test. P values were calculated by using two-sided paired or unpaired t tests, Wilcoxon matched-pairs signed rank test, Mann-Whitney test, repeated measures or ordinary one-way ANOVA (followed by Bonferroni's post hoc test) or Kruskal-Wallis test (followed by Dunn's post hoc test). Correlations were determined by simple linear regression. Statistical analysis was performed using Graphpad PRISM version 8.3.0 with significance set at P<0.05.

### REFERENCES

The superscripted numbers appearing in the present disclosure respectively correspond to the following references:
1 Armitage, J. O., Gascoyne, R. D., Lunning, M. A. & Cavalli, F. Non-Hodgkin lymphoma. Lancet 390, 298-310, doi:10.1016/S0140-6736(16)32407-2 (2017).
2 Hallek, M. Chronic lymphocytic leukemia: 2020 update on diagnosis, risk stratification and treatment. Am J Hematol 94, 1266-1287, doi:10.1002/ajh.25595 (2019).
3 Dreger, P. et al. Allogeneic stem cell transplantation provides durable disease control in poor-risk chronic lymphocytic leukemia: long-term clinical and MRD results of the German CLL Study Group CLL3X trial. Blood 116, 2438-2447, doi:10.1182/blood-2010-03-275420 (2010).
4 van Bruggen, J. A. C., Martens, A. W. J., Tonino, S. H. & Kater, A. P. Overcoming the Hurdles of Autologous T-Cell-Based Therapies in B-Cell Non-Hodgkin Lymphoma. Cancers (Basel) 12, doi:10.3390/cancers12123837 (2020).
5 Fraietta, J. A. et al. Determinants of response and resistance to CD19 chimeric antigen receptor (CAR) T cell therapy of chronic lymphocytic leukemia. Nat Med 24, 563-571, doi:10.1038/s41591-018-0010-1 (2018).
6 Slaney, C. Y., Wang, P., Darcy, P. K. & Kershaw, M. H. CARs versus BiTEs: A Comparison between T Cell-Redirection Strategies for Cancer Treatment. Cancer Discov 8, 924-934, doi:10.1158/2159-8290.CD-18-0297 (2018).
7 Haas, C. et al. Mode of cytotoxic action of T cell-engaging BiTE antibody MT110. Immunobiology 214, 441-453, doi:10.1016/j.imbio.2008.11.014 (2009).
8 Martens, A. W. J. et al. CD3xCD19 DART molecule treatment induces non-apoptotic killing and is efficient against high-risk chemotherapy and venetoclax-resistant chronic lymphocytic leukemia cells. J Immunother Cancer 8, doi:10.1136/jitc-2019-000218 (2020).
9 Topp, M. S. et al. Safety and activity of blinatumomab for adult patients with relapsed or refractory B-precursor acute lymphoblastic leukaemia: a multicentre, single-arm, phase 2 study. Lancet Oncol 16, 57-66, doi:10.1016/S1470-2045(14)71170-2 (2015).
10 Topp, M. S. et al. Long-term follow-up of hematologic relapse-free survival in a phase 2 study of blinatumomab in patients with MRD in B-lineage ALL. Blood 120, 5185-5187, doi:10.1182/blood-2012-07-441030 (2012).
11 Portell, C. A., Wenzell, C. M. & Advani, A. S. Clinical and pharmacologic aspects of blinatumomab in the treatment of B-cell acute lymphoblastic leukemia. Clin Pharmacol 5, 5-11, doi:10.2147/CPAA.542689 (2013).
12 Avery, D. T. et al. BAFF selectively enhances the survival of plasmablasts generated from human memory B cells. J Clin Invest 112, 286-297, doi:10.1172/JCI18025 (2003).
13 O'Connor, B. P. et al. BCMA is essential for the survival of long-lived bone marrow plasma cells. J Exp Med 199, 91-98, doi:10.1084/jem.20031330 (2004).
14 Thompson, J. S. et al. BAFF binds to the tumor necrosis factor receptor-like molecule B cell maturation antigen and is important for maintaining the peripheral B cell population. J Exp Med 192, 129-135, doi:10.1084/jem.192.1.129 (2000).
15 Bossen, C. & Schneider, P. BAFF, APRIL and their receptors: structure, function and signaling. Semin Immunol 18, 263-275, doi:10.1016/j.smim.2006.04.006 (2006).
16 Laurent, S. A. et al. gamma-Secretase directly sheds the survival receptor BCMA from plasma cells. Nat Commun 6, 7333, doi:10.1038/ncomms8333 (2015).
17 Novak, A. J. et al. Expression of BCMA, TACI, and BAFF-R in multiple myeloma: a mechanism for growth and survival. Blood 103, 689-694, doi:10.1182/blood-2003-06-2043 (2004).
18 Tai, Y. T. et al. APRIL and BCMA promote human multiple myeloma growth and immunosuppression in the bone marrow microenvironment. Blood 127, 3225-3236, doi:10.1182/blood-2016-01-691162 (2016).
19 Frerichs, K. A. et al. Preclinical Activity of JNJ-7957, a Novel BCMAxCD3 Bispecific Antibody for the Treatment of Multiple Myeloma, Is Potentiated by Daratumumab. Clin Cancer Res 26, 2203-2215, doi:10.1158/1078-0432.CCR-19-2299 (2020).
20 Raje, N. et al. Anti-BCMA CAR T-Cell Therapy bb2121 in Relapsed or Refractory Multiple Myeloma. N Engl J Med 380, 1726-1737, doi:10.1056/NEJMoa1817226 (2019).
21 Chiu, A. et al. Hodgkin lymphoma cells express TACI and BCMA receptors and generate survival and proliferation signals in response to BAFF and APRIL. Blood 109, 729-739, doi:10.1182/blood-2006-04-015958 (2007).
22 Darce, J. R., Arendt, B. K., Wu, X. & Jelinek, D. F. Regulated expression of BAFF-binding receptors during human B cell differentiation. J Immunol 179, 7276-7286, doi:10.4049/jimmuno1.179.11.7276 (2007).
23 Ng, L. G. et al. B cell-activating factor belonging to the TNF family (BAFF)-R is the principal BAFF receptor facilitating BAFF costimulation of circulating T and B cells. J Immunol 173, 807-817, doi:10.4049/jimmuno1.173.2.807 (2004).
24 Dogan, A. et al. B-cell maturation antigen expression across hematologic cancers: a systematic literature review. Blood Cancer J 10, 73, doi:10.1038/s41408-020-0337-y (2020).
25 Lee, L. et al. Evaluation of B cell maturation antigen as a target for antibody drug conjugate mediated cytotoxicity in multiple myeloma. Br J Haematol 174, 911-922, doi:10.1111/bjh.14145 (2016).
26 Ferrer, G. et al. B cell activation through CD40 and IL4R ligation modulates the response of chronic lymphocytic leukaemia cells to BAFF and APRIL. Br J Haematol 164, 570-578, doi:10.1111/bjh.12645 (2014).
27 Friedman, K. M. et al. Effective Targeting of Multiple B-Cell Maturation Antigen-Expressing Hematological Malignances by Anti-B-Cell Maturation Antigen Chimeric Antigen Receptor T Cells. Hum Gene Ther 29, 585-601, doi:10.1089/hum.2018.001 (2018).
28 van Bruggen, J. A. C. et al. Chronic lymphocytic leukemia cells impair mitochondrial fitness in CD8(+) T cells and impede CAR T-cell efficacy. Blood 134, 44-58, doi:10.1182/blood.2018885863 (2019).
29 Ramsay, A. G., Clear, A. J., Fatah, R. & Gribben, J. G. Multiple inhibitory ligands induce impaired T-cell immunologic synapse function in chronic lymphocytic leukemia that can be blocked with lenalidomide: establishing a reversible immune evasion mechanism in human cancer. Blood 120, 1412-1421, doi:10.1182/blood-2012-02-411678 (2012).
30 Riches, J. C. et al. T cells from CLL patients exhibit features of T-cell exhaustion but retain capacity for cytokine production. Blood 121, 1612-1621, doi:10.1182/blood-2012-09-457531 (2013).
31 Walker, A. J. et al. Tumor Antigen and Receptor Densities Regulate Efficacy of a Chimeric Antigen Receptor Targeting Anaplastic Lymphoma Kinase. Mol Ther 25, 2189-2201, doi:10.1016/j.ymthe.2017.06.008 (2017).
32 Majzner, R. G. et al. Tuning the Antigen Density Requirement for CAR T-cell Activity. Cancer Discov 10, 702-723, doi:10.1158/2159-8290.CD-19-0945 (2020).
33 Pont, M. J. et al. gamma-Secretase inhibition increases efficacy of BCMA-specific chimeric antigen receptor T cells in multiple myeloma. Blood 134, 1585-1597, doi:10.1182/blood.2019000050 (2019).
34 Doody, R. S. et al. A phase 3 trial of semagacestat for treatment of Alzheimer's disease. N Engl J Med 369, 341-350, doi:10.1056/NEJMoa1210951 (2013).
35 Panza, F. et al. REVIEW: gamma-Secretase inhibitors for the treatment of Alzheimer's disease: The current state. CNS Neurosci Ther 16, 272-284, doi:10.1111/j.1755-5949.2010.00164.x (2010).
36 Tolcher, A. W. et al. Phase I study of R04929097, a gamma secretase inhibitor of Notch signaling, in patients with refractory metastatic or locally advanced solid tumors. J Clin Oncol 30, 2348-2353, doi:10.1200/JCO.2011.36.8282 (2012).
37 Krop, I. et al. Phase I pharmacologic and pharmacodynamic study of the gamma secretase (Notch) inhibitor MK-0752 in adult patients with advanced solid tumors. J Clin Oncol 30, 2307-2313, doi:10.1200/JCO.2011.39.1540 (2012).
38 Messersmith, W. A. et al. A Phase I, dose-finding study in patients with advanced solid malignancies of the oral gamma-secretase inhibitor PF-03084014. Clin Cancer Res 21, 60-67, doi:10.1158/1078-0432.CCR-14-0607 (2015).
39 Pozzo, F. et al. NOTCH1-mutated chronic lymphocytic leukemia cells are characterized by a MYC-related overexpression of nucleophosmin 1 and ribosome-associated components. Leukemia 31, 2407-2415, doi:10.1038/1eu.2017.90 (2017).
40 Kridel, R. et al. Whole transcriptome sequencing reveals recurrent NOTCH1 mutations in mantle cell lymphoma. Blood 119, 1963-1971, doi:10.1182/blood-2011-11-391474 (2012).
41 Josefsson, S. E. et al. TIGIT and PD-1 Mark Intratumoral T Cells with Reduced Effector Function in B-cell Non-Hodgkin Lymphoma. Cancer Immunol Res 7, 355-362, doi:10.1158/2326-6066.CIR-18-0351 (2019).
42 Ramsay, A. G. et al. Follicular lymphoma cells induce T-cell immunologic synapse dysfunction that can be repaired with lenalidomide: implications for the tumor microenvironment and immunotherapy. Blood 114, 4713-4720, doi:10.1182/blood-2009-04-217687 (2009).
43 Tonino, S. H. et al. Expansion of effector T cells associated with decreased PD-1 expression in patients with indolent B cell lymphomas and chronic lymphocytic leukemia. Leuk Lymphoma 53, 1785-1794, doi:10.3109/10428194.2012.673224 (2012).
44 Hilchey, S. P. et al. Follicular lymphoma tumor-infiltrating T-helper (T(H)) cells have the same polyfunctional potential as normal nodal T(H) cells despite skewed differentiation. Blood 118, 3591-3602, doi:10.1182/blood-2011-03-340646 (2011).
45 Bird, C. H. et al. The granzyme B-Serpinb9 axis controls the fate of lymphocytes after lysosomal stress. Cell Death Differ 21, 876-887, doi:10.1038/cdd.2014.7 (2014).
46 Jiang, P. et al. Signatures of T cell dysfunction and exclusion predict cancer immunotherapy response. Nat Med 24, 1550-1558, doi:10.1038/s41591-018-0136-1 (2018).
47 Fritsch, K., Finke, J. & Grullich, C. Suppression of granzyme B activity and caspase-3 activation in leukaemia cells constitutively expressing the protease inhibitor 9. Ann Hematol 92, 1603-1609, doi:10.1007/s00277-013-1846-6 (2013).
48 Ben Safta, T. et al. Granzyme B-activated p53 interacts with Bcl-2 to promote cytotoxic lymphocyte-mediated apoptosis. J Immunol 194, 418-428, doi:10.4049/jimmunol.1401978 (2015).
49 Meslin, F., Thiery, J., Richon, C., Jalil, A. & Chouaib, S. Granzyme B-induced cell death involves induction of p53 tumor suppressor gene and its activation in tumor target cells. J Biol Chem 282, 32991-32999, doi:10.1074/jbc.M705290200 (2007).
50 Johnsrud, A. J. et al. Infectious and immunological sequelae of daratumumab in multiple myeloma. Br J Haematol 185, 187-189, doi:10.1111/bjh.15433 (2019).

What is claimed:

1. A method for treating non-Hodgkin lymphoma (NHL) in a human subject comprising administering to the subject a therapeutically effective amount of a BCMA-specific antibody.

2. The method of claim 1, wherein the BCMA-specific antibody is monospecific or bispecific.

3. The method of claim 1 wherein the BCMA-specific antibody is teclistamab.

4. The method of claim 1 wherein the non-Hodgkin lymphoma is a subtype characterized by expression of B cell maturation antigen (BCMA).

5. The method of claim 1 wherein the non-Hodgkin lymphoma is chronic lymphocytic leukemia (CLL), lymphoblastic lymphoma, mantle cell lymphoma, diffuse large B-cell lymphoma, or Waldestrom macroglobulinemia

6. The method of claim 1 wherein the subject is an adult.

7. The method of claim 1, wherein the amount of teclistamab that is administered to the subject is effective to activate T cells in the subject, induce neutrophil degranulation in the subject, induce cytokine production in the subject, or any combination thereof

8. The method of claim 1, further comprising administering to the subject a γ-secretase inhibitor.

9. The method according to claim 8, wherein the BCMA-specific antibody and the γ-secretase inhibitor are administered to the subject in a single dosage form.

10. The method according to claim 8, wherein the BCMA-specific antibody is administered in a first dosage form and the γ-secretase inhibitor is administered in a second dosage form.


SEQUENCE LISTING (21 sequences)

SEQ ID NO: 1 - protein, 7 aa, Artificial Sequence
  BCMB69 HCDR1
SGSYFWG

SEQ ID NO: 2 - protein, 16 aa, Artificial Sequence
  BCMB69 HCDR2
SIYYSGITYYNPSLKS

SEQ ID NO: 3 - protein, 11 aa, Artificial Sequence
  BCMB69 HCDR3
HDGAVAGLFDY

SEQ ID NO: 4 - protein, 11 aa, Artificial Sequence
  BCMB69 LCDR1
GGNNIGSKSVH

SEQ ID NO: 5 - protein, 7 aa, Artificial Sequence
  BCMB69 LCDR2
DDSDRPS

SEQ ID NO: 6 - protein, 11 aa, Artificial Sequence
  BCMB69 LCDR3
QVWDSSSDHVV

SEQ ID NO: 7 - protein, 121 aa, Artificial Sequence
  BCMB69 VH
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 8 - protein, 111 aa, Artificial Sequence
  BCMB69 VL
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQP

SEQ ID NO: 9 - protein, 448 aa, Artificial Sequence
  BCMB69 HC
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  BCMB69 LC
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 11 - protein, 5 aa, Artificial Sequence
  CD3B219 HCDR1
TYAMN

SEQ ID NO: 12 - protein, 19 aa, Artificial Sequence
  CD3B219 HCDR2
RIRSKYNNYATYYAASVKG

SEQ ID NO: 13 - protein, 14 aa, Artificial Sequence
  CD3B219 HCDR3
HGNFGNSYVSWFAY

SEQ ID NO: 14 - protein, 14 aa, Artificial Sequence
  CD3B219 LCDR1
RSSTGAVTTSNYAN

SEQ ID NO: 15 - protein, 7 aa, Artificial Sequence
  CD3B219 LCDR2
GTNKRAP

SEQ ID NO: 16 - protein, 9 aa, Artificial Sequence
  CD3B219 LCDR3
ALWYSNLWV

SEQ ID NO: 17 - protein, 125 aa, Artificial Sequence
  CD3B219 VH
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 18 - protein, 112 aa, Artificial Sequence
  CD3B219 VL
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 19 - protein, 452 aa, Artificial Sequence
  CD3B219 HC
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 215 aa, Artificial Sequence
  CD3B219 LC
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 327 aa, Artificial Sequence
  wild-type IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK